(19) United States
(12) Patent Application Publication
(10) Pub. No.: US 2007/0190089 A1
(43) Pub. Date: Aug. 16, 2007

(54) ANTIPERSPIRANTS WITH WATER SOLUBLE ESTERS

(76) Inventors: Leonard Mackles, New York, NY (US); William Bess, Edison, NJ (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/670,583

(22) Filed: Feb. 2, 2007

Related U.S. Application Data
(60) Provisional application No. 60/765,895, filed on Feb. 7, 2006.

(51) Int. Cl. A61K 8/28; A61K 8/26

(52) U.S. Cl. 424/401; 424/65; 424/66; 424/68

(57) ABSTRACT

This invention provides antiperspirants to combat axillary body odor by preventing or reducing perspiration, wherein the antiperspirant comprises a water-soluble ester to reduce the problems associated with stickiness and residue. The present invention provides a new class of detackifying agents that are easily formulated into clear, aqueous based antiperspirant formulations. These agents are readily water soluble, provide clear cosmetically elegant products and eliminate the stickiness and tackiness normally associated with aqueous based products.

### FIELD OF INVENTION

This invention relates to antiperspirants to combat axillary body odor by preventing or reducing perspiration, wherein the antiperspirant comprises a water-soluble ester to reduce the problems associated with stickiness and residue.

1. Background of the Invention

Antiperspirants combat axillary body odor by preventing or reducing perspiration via the action of aluminum and or zirconium astringent salts. Deodorants combat these odors by either attacking the microbes that degrade perspiration into malodorous compounds, or by covering the odor via perfumes. Antiperspirants are often effective deodorants as well since the astringent salts can be effective antimicrobial agents. For this reason, antiperspirants are often preferred to deodorants since they prevent or reduce the amount of wetness as well as reducing or eliminating malodors.

In order to act effectively, antiperspirant salts must be utilized in high concentration. When utilized in effective concentrations, these salts become sticky as they dry and produce an unpleasant tackiness in the underarm area. Numerous formulations have been developed over the years to address this problem. Emulsion based creams were developed that exhibited reduced stickiness, but left an unsightly residue on the skin and clothing. Emulsion based roll-ons were an improvement over creams in that they could be applied in a thinner film, but they did not reduce the stickiness, and were slow drying. Aerosol sprays based on fatty esters were developed. These provided relief from the stickiness but produced a dusty spray around the user, often resulting in a choking feeling. In addition, the fatty acid esters often reduced antiperspirant efficacy by coating the antiperspirant salt particle with a water insoluble film. The esters also promoted staining of the clothing around the underarm area. The staining was aggravated by the fact that these esters are not water soluble, and therefore once on the clothing, are not easily washed off. Additionally, environmental issues arose around the use of the fluorocarbon propellants utilized. More recent aerosol formulations have been developed with non-fluorocarbon propellants, but these have storage issues regarding flammability and explosion hazard, as well as the other issues with aerosols described above.

Volatile silicone suspensions of antiperspirant salts have been marketed in roll-on and solid stick forms. While successfully addressing the stickiness issue, these forms leave a white powdery residue on the skin and clothing. More recently clear antiperspirant gels have been marketed which reduce or eliminate the white powdery residue, but these have not satisfactorily reduced the stickiness issue.

The development of a clear, non-sticky, non-residue producing antiperspirant remains a desired goal. Several attempts to develop these types of products have been disclosed in the prior art. U.S. Pat. No. 5,376,363 (Benfatto et al.) discloses clear or translucent gelled antiperspirant sticks which are substantially anhydrous and utilize a gelling agent such as dibenzylidene acetal. However, these types of products suffer from an inherent stickiness due to the combination of the gelling agent and antiperspirant salt. U.S. Pat. No. 5,833,963 (Mackles et al.) discloses the use of polyglycols as detackifying agents in water based antiperspirant formulations, but does not disclose the use of esters as detackifying agents in water based antiperspirant compositions.

2. Description of Related Art

U.S. Pat. No. 4,548,808 (Chavkin) discloses the use of triacetin (glycerol triacetate) in anhydrous suspension type antiperspirant products to give long lasting deodorant activity. While chemically related to glycerol diacetate, triacetin has only very limited water solubility, and does not reduce the stickiness of water based antiperspirant formulations.

U.S. Pat. No. 4,346,079 (Roehl) discloses an example of an anhydrous dibenzylidene monosorbitol acetal based solid antiperspirant. In the example a small amount of glycerol diacetate is utilized. However, no mention of its use as a detackifying agent is made anywhere in the patent disclosure.

The above references do not teach or anticipate the use of these esters as detackifying agents in water based antiperspirant compositions. As such, someone skilled in the art could not anticipate the use of these esters for detackifying water based antiperspirants based on the disclosed prior art.

Low molecular weight water soluble acids such as acetic acid are known to have the ability to reduce the growth of microorganisms on the skin. Without in any way restricting the present invention, it is believed that esterases elaborated by microorganisms on the skin hydrolyze the low molecular weight water soluble ester, thereby releasing the water soluble organic acid The resulting lowered pH inhibits the growth of the microorganisms and chemically neutralizes odorous products of microorganisms and of the action of bacterial esterases on the apocrine secretions in the axilla. The ability of microorganisms to produce odor is thus reduced. Although the skin's natural buffering capacity eventually neutralizes these acids and esterases become active, the hydrolysis of the remaining esters on the skin again lowers the skin pH to inactivate the esterases and prevent or delay the development of odor.

All references cited herein are incorporated herein by reference in their entireties.

### BRIEF SUMMARY OF THE INVENTION

The present invention provides a new class of detackifying agents that are easily formulated into clear, aqueous based antiperspirant formulations. These agents are readily water soluble, provide clear cosmetically elegant products and eliminate the stickiness and tackiness normally associated with aqueous based products.

### DETAILED DESCRIPTION OF PREFERRED EMBODIMENTS OF THE INVENTION

The present invention utilizes a class of water-soluble short chain esters having water solubility of at least 10% at 25 C. These water soluble short chain esters have unexpected effect of eliminating stickiness from a water based antiperspirant. This class contains all esters that are composed of a water-soluble (5% or greater at 25 C) alcohol, glycol or polyol and a water-soluble (5% or greater at 25 C) organic acid. Non-limiting examples are glycerol monoacetate, ethylene glycol diacetate and glycerol diacetate. Since these esters are water soluble, they do not adversely affect staining properties, and do not adversely affect antiperspirant efficacy. Additionally, formulations utilizing these agents are easily manufactured with simple mixing, thus avoiding the complex manufacturing processes often required to manufacture antiperspirant products.

The invention provides a stable, aqueous-based antiperspirant composition containing water soluble esters which very effectively delivers non-tacky, water-soluble antiperspirant active materials to the skin for use in combating perspiration and body malodor, e.g., in axillary regions of the human body, by applying the antiperspirant compositions to the human body, e.g., to the skin in axillary regions of the body.

The invention provides cosmetic compositions of antiperspirants comprising water soluble esters which do not exhibit tackiness, which feel uniform and dry after application, do not feel greasy or oily, are applied clear to the skin and remain clear and clean after application, regardless of the forms in which the antiperspirant compositions are delivered (e.g., roll-on, stick, or spray).

The invention provides an aqueous-based antiperspirant composition comprising a water soluble ester for use in methods for treating or preventing human malodor associated with perspiration and sweating, especially underarm malodor. The methods comprise applying to the skin of a human a safe and effective amount of the antiperspirant compositions of the invention.

The invention provides an aqueous-based antiperspirant solution technology utilizing water soluble esters that minimizes or eliminates tack. In accordance with the invention the antiperspirant solution technology is suitable for a wide variety of different delivery forms, has acceptable and advantageous organoleptic qualities, and costs significantly less than currently available antiperspirant products. The solution technology of the invention is also economical and easy to manufacture.

It will be appreciated by those having skill in the art that the aqueous-based antiperspirant compositions of the invention are effective and useful as common, aqueous-based solution formulations which may in turn be formulated into a variety of cosmetic dosage and/or delivery forms for application and administration of antiperspirant active materials. Examples of the forms in which the aqueous-based antiperspirants may be manufactured and/or produced include, but are not limited to, creams, gels, solids (e.g. barrier pack; liquisticks), sprays (e.g. pump sprays), aerosols, roll-ons, opacified roll-ons, foams, and the like. Although not a requirement for use, the preferred modes of applying the antiperspirant compositions of the invention are roll-ons, sprays, aerosols, and gels.

The compositions may also include other ingredients that are normally employed in antiperspirant formulations. Examples of other ingredients that may be included in the compositions of the instant invention include but are not limited to thickeners, opacifiers, antimicrobial agents, emulsifiers, additional emollients, fragrances, gelling agents, co-solvents, surfactants, alcohols, glycols, silica, talc, coloring agents, and the like.

The invention will be illustrated in more detail with reference to the following Examples, but it should be understood that the present invention is not deemed to be limited thereto.

### EXAMPLES

There is given in tabular form below Examples of the invention in which the vehicle is primarily water in which the composition components are dissolved.

### Example 1

### Antiperspirant Roll-On

An antiperspirant composition of the invention was prepared by mixing the ingredients as presented in this Example.

| Ingredient | % W/W |
|---|---|
| Aluminum Chlorohydrate 50% sol'n | 40.00 |
| Glyceryl Diacetate | 35.00 |
| Tween 80 | 1.00 |
| Perfume | 0.10 |
| Water | 23.90 |
| | 100.00 |

### Example 2

### Antiperspirant Roll-On

An antiperspirant composition of the invention was prepared by mixing the ingredients as presented in this Example.

| Ingredient | % W/W |
|---|---|
| Aluminum-Zirconium Tetrachlorohydrex- | 60.00 |
| Gly 35% sol'n | |
| Glyceryl Diacetate | 30.00 |
| Tween 80 | 1.00 |
| Perfume | 0.10 |
| Water | 8.90 |
| | 100.00 |

### Example 3

### Antiperspirant Pump-Spray

An antiperspirant composition of the invention was prepared by mixing the ingredients as presented in this Example.

| Ingredient | % W/W |
|---|---|
| Aluminum Chlorohydroxide 50% sol'n | 24.00 |
| Glyceryl Diacetate | 28.00 |
| Tween 80 | 1.00 |
| Perfume | 0.10 |
| Water | 46.90 |
| | 100.00 |

While the invention has been described in detail and with reference to specific examples thereof, it will be apparent to one skilled in the art that various changes and modifications can be made therein without departing from the spirit and scope thereof.

What is claimed is:

1. An antiperspirant composition comprising:
an antiperspirant active ingredient;
a water soluble ester comprising a water soluble alcohol, glycol or polyol and a water soluble organic acid, in an amount of from 1 to 50% by weight of the composition; and
water in an amount to equal 100% by weight of the composition.

2. The antiperspirant composition of claim 1, wherein the antiperspirant active ingredient is selected from the group consisting of aluminum, zirconium, and aluminum zirconium salts.

3. An antiperspirant composition comprising:
an antiperspirant active ingredient;
a water soluble ester in an amount of from 1 to 50% by weight of the composition; and
water in an amount to equal 100% by weight of the composition.

4. The antiperspirant composition as in any one of claims 1, 2, or 3, wherein the water soluble ester comprises a water soluble alcohol, glycol or polyol and a water soluble organic acid.

5. The antiperspirant composition as in any one of claims 1, 2, or 3, in stick form.

6. The antiperspirant composition as in any one of claims 1, 2, or 3, in roll-on form.

7. The antiperspirant composition as in any one of claims 1, 2, or 3, in spray form.

8. The antiperspirant composition as in any one of claims 1, 2, or 3, wherein the water soluble ester is glyceryl monoacetate.

9. The antiperspirant composition as in any one of claims 1, 2, or 3, wherein the water soluble ester is glyceryl diacetate.

10. The antiperspirant composition as in any one of claims 1, 2, or 3, wherein the water soluble ester is a mixture of glyceryl monoacetate and glyceryl diacetate.

11. A method of producing an antiperspirant composition comprising:
admixing an antiperspirant active ingredient with a water soluble ester in an amount of from 1 to 50% by weight of the composition, and adding water in an amount to equal 100% by weight of the composition.

12. The method of claim 11, wherein the antiperspirant active ingredient is selected from the group consisting of aluminum, zirconium, and aluminum zirconium salts.

13. The method of claim 11, wherein the water soluble ester comprises a water soluble alcohol, glycol or polyol and a water soluble organic acid.

14. A method for treating or preventing human malodor associated with perspiration and sweating, especially underarm malodor, comprising:
applying to the axillary region of a human an antiperspirant composition comprising
an antiperspirant active ingredient;
a water soluble ester comprising a water soluble alcohol, glycol or polyol and a water soluble organic acid, in an amount of from 1 to 50% by weight of the composition; and
water in an amount to equal 100% by weight of the composition.

15. The method of claim 14, wherein the antiperspirant active ingredient is selected from the group consisting of aluminum, zirconium, and aluminum zirconium salts.
